(19) United States
(12) Patent Application Publication
(10) Pub. No.: US 2023/0290497 A1
(43) Pub. Date: Sep. 14, 2023

(54) IMPLANTABLE SENSOR DEVICE

(71) Applicant: IX Innovation LLC, Seattle, WA (US)

(72) Inventors: Jeffrey Roh, Seattle, WA (US); Justin Esterberg, Mercer Island, WA (US); John Cronin, Jericho, VT (US); Seth Cronin, Essex Junction, VT (US); Michael J. Baker, Georgia, VT (US)

(73) Assignee: IX Innovation LLC, Seattle, WA (US)

(21) Appl. No.: 17/691,600

(22) Filed: Mar. 10, 2022

(51) Int. Cl. G16H 40/67; G16H 40/40; G16H 10/60

(52) U.S. Cl. CPC G16H 40/67; G16H 10/60; G16H 40/40

(57) ABSTRACT

An implantable sensor device for monitoring patient conditions surrounding a surgical site is disclosed. The implantable sensor device comprises one or more sensors, a memory, and a controller. The implantable sensor device is configured to confirm activation of the implantable sensor device. Further, the implantable sensor device monitors the one or more sensors sensing the implanted device and stores the monitored data in the memory and sends the monitored data to the external device by establishing a connection with the external device, via a network interface. The implantable sensor device is further configured to monitor the stored data for the adverse event and perform an action to mitigate the risk of the life of the patient caused due to the adverse event.

### FIELD OF THE DISCLOSURE

The present disclosure is generally related to sensor devices for medical patients, and more particularly related to an implantable sensor device for monitoring patient conditions surrounding a surgical site.

### BACKGROUND

With the advancement in medical device technology, especially in microelectronics and biotechnology, many implantable medical devices such as the pacemakers, implantable cardioverter defibrillators (ICDs), coronary stents, hip implants, and insulin pumps have been developed. Such implantable medical devices are often used to sense physiological responses of a patient’s body in vivo, or to actuate physiological organs. However, it is difficult to monitor the condition of the implanted medical device inside the patient’s body for degradation, deformation, or the condition of the surrounding tissue in presence of the implanted medical device. Thus, to monitor and discover the condition of the in-vivo implanted medical device, medical professionals rely on medical imagery to view the surgical sites or, if necessary, perform a surgical operation to directly access the site.

In order to avoid the surgical operation, implantable sensor devices have been developed. The development of the implantable sensor devices preceded the modern medical device industry boom that happened in the 1960’s.

However, the existing implantable sensor devices are not smart enough to take an action in order to mitigate the risk of harm to the patient if an adverse condition is detected based on data received from the biomedical sensor. In addition, the existing implantable sensor devices are unable to monitor recovery of the patient after surgery. The existing implantable sensor devices are also unable to provide the real-time assistance to the patient by notifying the condition to the patient’s surgeon or caregiver. Thus, the existing devices carry the risk of a delayed action which could be crucial in patient’s critical conditions.

As such, the medical device industry is in need of an implantable sensor device that can overcome all the aforementioned limitations and provide an efficient and reliable implantable sensor device that can mitigate the risk of life based on the data received from the biomedical sensor.

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

### SUMMARY

In an embodiment an implantable sensor device is provided. The implantable sensor device includes an implantable sensor including at least one sensor, a memory, a controller, and a base module, a network interface; and an action module. The base module is configured to sense a physical attribute of the patient with the at least one sensor to collect monitored data; store the monitored data in the memory; monitor the stored data for an adverse event; and perform an action in response to the adverse event.

In some aspects, the techniques described herein relate to a implantable sensor device, wherein the base module is further configured to confirm activation of the implantable sensor device.

In accordance with aspects of the present disclosure, the base module can be further configured to establish a data connection with an external device, via the network interface. The network interface can be configured to send the monitored data to the external device, wherein the monitored data is sent based on the configuration of the implantable sensor device. The data connection can be a wireless network connection between the implantable sensor device and the external device. The base module is configured to send a control signal to an action module to mitigate an impact of the adverse event. The action module can be configured to perform a medical procedure on the patient to mitigate the impact of the adverse event. The base module can be configured to send the monitored data, in real-time, via the network interface to an external device. The at least one sensor can be at least one of a force transducer, a strain gauge, a pulse oximeter, electromyography sensor, electrocardiography sensor, temperature sensor, pH sensor, blood glucose sensor, or an accelerometer. The at least one sensor can include two or more sensors of different types.

In accordance with an embodiment of the present disclosure, a system for monitoring implant conditions in vivo is provided. The system includes an implantable medical device; an external monitoring computing device; and an implantable sensor, disposed on the implantable device, including at least one sensor, a memory, a controller, and a base module. The base module is configured to: monitor the implantable sensor with the at least one sensor to collect monitored data; store the monitored data in the memory; monitor the stored data for an adverse event; and perform an action in response to the adverse event.

In accordance with aspects of the instant disclosure, the base module can further include a network interface, and the base module is further configured to establish a data connection with the external monitoring computing device, via the network interface, to report the monitored conditions of the implanted device. The data connection can be a wireless network connection between the implantable sensor and the external monitoring computing device. The base module can be configured to send a control signal to an action module to mitigate an impact of the adverse event. The action module can be configured to perform a medical procedure on the patient to mitigate the impact of the adverse event. The base module can be configured to send the monitored data, in real-time, to the external monitored computing device. The at least one sensor can be at least one of a force transducer, a strain gauge, a pulse oximeter, electromyography sensor, electrocardiography sensor, temperature sensor, pH sensor, blood glucose sensor, or an accelerometer. The at least one sensor can include two or more sensors of different types. The at least one sensor is biodegradable.

In accordance with embodiments of the present disclosure, a method of in vivo monitoring is provided. The method includes implanting an implantable sensor in a patient, the implantable sensor including at least one sensor, a memory, a controller, and a base module, sensing at least one physical attribute of the patient with the at least one sensor to collect monitored data; storing the monitored data in the memory; monitoring the stored data for an adverse event; and performing a corrective action in response to the adverse event.

In accordance with aspects of the instant disclosure, the implanting step can include implanting an implanted device and the implantable sensor can be disposed on the implanted device. The implanting step can include implanting a redundant implanted device and the performing the corrective action step includes deactivating the implanted device and activating the redundant implanted device. The sensing step can include sensing at least two physical attributes of the patient. The method can further include identify priority of the at least two physical attributes of the patient based upon a pre-defined priority order and prioritizing a higher priority sensed physical attribute over a lower priority sensed physical attribute. The higher priority sensed physical attribute can be sent to an external computing device. The method can further include establishing a wireless data connection with an external monitoring computing device, via a network interface, to report the monitored data. The corrective action can include performing a medical procedure on the patient to mitigate the impact of the adverse event. The method can further include send the monitored data, in real-time, to an external monitored computing device. The at least one sensor is at least one of a force transducer, a strain gauge, a pulse oximeter, electromyography sensor, electrocardiography sensor, temperature sensor, pH sensor, blood glucose sensor, or an accelerometer. The at least one sensor can include two or more sensors of different types.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1A illustrates a block diagram of an implantable sensor device, according to an embodiment;

FIG. 1B illustrates a partial side view of an implantable sensor device and an implant disposed in a patient according to an embodiment;

FIG. 2 illustrates a patient database to store data for the implantable sensor device, according to an embodiment;

FIG. 3A illustrates a flow chart showing a method of operation of a base module in the system, according to an embodiment;

FIG. 3B illustrates a flow chart showing the method of operation of a base module in the system, according to an embodiment;

FIG. 4 illustrates a flow chart showing a method of operation of a boot-up module in the system, according to an embodiment;

FIG. 5 illustrates a flow chart showing a method of operation of a monitoring module in the system, according to an embodiment;

FIG. 6 illustrates a flow chart showing a method of operation of a communication module in the system, according to an embodiment; and

FIG. 7 illustrates a flow chart showing a method of operation of an action module in the system, according to an embodiment.

### DETAILED DESCRIPTION

Some embodiments of this disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items or meant to be limited to only the listed item or items.

It must also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, the preferred, systems and methods are now described.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The instant implantable sensor devices, disclosed herein, are designed to take an action in order to mitigate the risk of harm to the patient if an adverse condition is detected based on data received from the implantable, biomedical, sensor devices. In addition, the instant implantable sensor devices are able to monitor recovery of the patient after surgery. Further, the instant implantable sensor devices are also able to provide real-time assistance to the patient by notifying the condition to the patient’s surgeon or caregiver. Thus, the instant devices resolve many of the risks of a delayed action which are crucial in patient’s critical conditions.

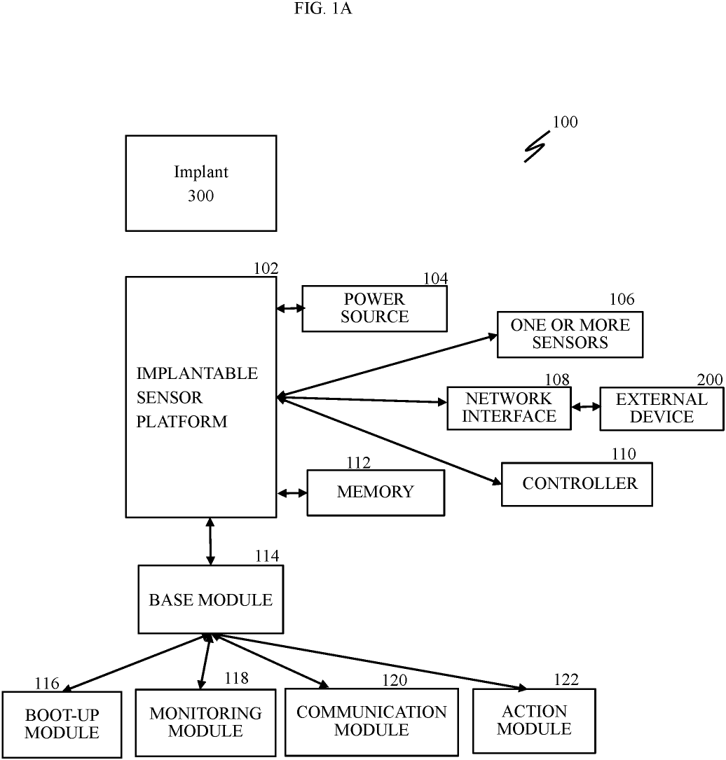

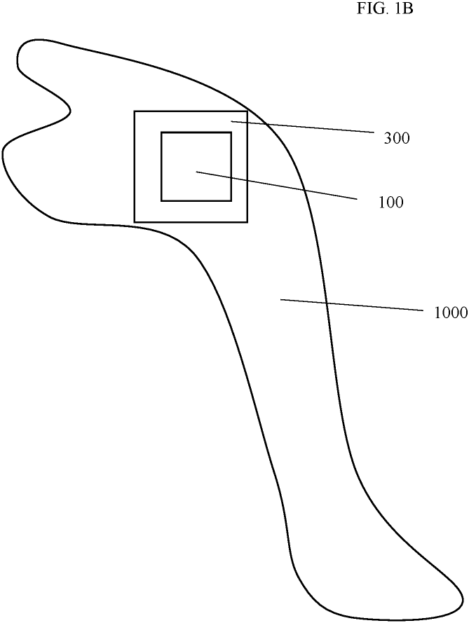

In accordance with a first preferred embodiment, FIG. 1A illustrates schematic of an implantable sensor device 100. The implantable sensor device 100 can include, among other things, an implantable sensor platform 102. The implantable sensor platform 102 can be a general-purpose computing system which can include one or more sensors 106 to perform functions based on the patient’s health requirement. In addition, the implantable sensor platform 102 may also enable the use of several other integrated sensors 106. In some embodiments, the implantable sensor platform 102 can monitor a patient’s health condition and can send notifications of a health condition to an external device for real-time reporting to a user or a doctor. Real-time reporting can be dependent on the specific measurements acquired by the sensors, such that the measured data can be reported immediately following acquisition of the measurement or transmitting at the time of measurement with negligible latency. Each measurement can be transmitted to a receiving device, such as the external device 200, with each transmitted measurement being received by the receiving device with no delay, or the smallest delay possible resulting from latency inherent in a network. Alternatively, or additionally, the implantable sensor platform 102 can check at least one health parameter of the patient for a threshold value. On reaching the threshold value, the implantable sensor platform 102 can take an action to modify the function, such as but not limited to turn off the implantable sensor device 100 for a period of time; turn off the implantable sensor device 100 until receiving a reactivating signal; send a real time notification to user device or network; or record adverse event. For example, if the implantable sensor platform 102 senses that a patient’s blood pressure exceeds a desired value, e.g., is too high or too low, the device can distribute medication to correct the unacceptable blood pressure readings. Additionally, or alternatively, the implantable sensor platform 102 can contact the patient’s medical provider to schedule a follow up appointment or message the patient that emergency medical care is required.

In general, the implantable sensor platform 102 can be coupled to a power source 104, one or more sensors 106, a network interface 108, a controller 110, and a memory 112. The implantable sensor platform 102 can be configured to run a base module 114 which can be software or a non-transitory computer readable medium running software, which can include a number of software modules including, but not limited to, a boot-up module 116, a monitoring module 118, a communication module 120, and an action module 122. The aforementioned modules can include instructions for operating the hardware sensors 106, network interface 108, controller 110, memory 112, other hardware features. The various hardware and software modules can work in concert to allow for the implantable sensor platform 102 to sense relevant in vivo conditions of a patient and, if necessary, perform prophylactic actions internal to the patient to prevent further damage to the surgical site or prevent other health conditions.

The one or more sensors 106 may be implantable as part of the implantable sensor platform 102. The implantable sensors 106 can measure one or more physical metrics, or attributes, related to the health of a patient, or the status of an implant or part of the patient’s body. The one or more sensors 106 can include, but not limited to, sensors which are capable of measuring an electrical activity of nervous tissues, tissue health, activity of a muscle, integrity of an implant. For example, the one or more sensors 106 can be one or a combination of force transducers, strain gages, pulse oximeters, electromyography sensors, electrocardiography sensors, temperature sensors, pH sensors, blood glucose sensors, and accelerometers. The one or more sensors 106 may be integrated, directly, into the implantable sensor device 100 on an implant 300 to monitor the device itself and can include sensors such as force transducers and strain gages to monitor the structural integrity of an implant 300. This monitored data may be used to anticipate a premature failure of an installed implant 300.

For example, as seen in FIG. 1B, the one or more sensors 106 of the implantable sensor device 100 may, alternatively, be disposed on or in the implant 300 and can be used to monitor a region within the patient 1000, such as monitoring temperature and pH for signs of infection. In some embodiments, the temperature sensor can be a graphene sensors which can use low current and low voltage to measure temperature changes via a change in resistance. These sensors 106 may be integrated into the implanted device 300 or may be independent of such implanted devices 300. The one or more sensors 106 may also be able to monitor a patient’s 1000 movements, such as by incorporating accelerometers, to detect the position of a limb of the person. For example, in the case of a knee replacement, partial or total, the implant 300, i.e., the knee replacement, can include a plurality of sensors 106 including a temperature and pH sensor for detecting signs of post implantation infection. The plurality of sensors 106 can additionally include at least one accelerometer to monitor proper kinematics of the knee during rehabilitation. Multiple accelerometers may be located at different locations, such as at multiple points on an arm or leg, to be able to detect the relative position of the parts of the leg. Such sensors 106 could be used to monitor a patient’s range of motion or to detect a fall or a limb extending beyond a normal range of motion to predict a possible injury. Thus, the implantable sensor device 100 can monitor the conditions surrounding the implantation site in the patient 1000 and/or monitor conditions of the implant 300 itself. Alternatively, such a system can be implemented with other implants, e.g., hip replacements, spinal implants, or other implants.

Further, in some embodiments, the one or more sensors 106 may be made of biodegradable material, to facilitate easy disposal of the one or more sensors 106 once implanted inside the patient’s body. It can be noted that the one or more sensors 106 may be comprised of biodegradable materials designed to work for a specified period of time and get dissolved harmlessly and completely into the body’s own fluids. Further, the one or more sensors 106 may be used to determine the heart rate, SpO2, blood pressure, blood flow volume or perfusion, of the patient.

In an embodiment, the controller 110 may include one or more microprocessors, microcontrollers, digital signal processors, central processing units, logic circuitries, and/or any devices that can perform instructions stored in the memory 112. In one case, the controller 110 may execute instructions or programs or perform calculations on the data received from the one or more sensors 106. In another example, the controller 110 may execute instructions or programs or perform calculations on the data stored in the patient database, such as the base module 114, boot-up module 116, the monitoring module 118, the communication module 120, and the action module 122.

The memory 112 can store one or more instructions and monitored data, including sensor data or software modules 114, 116, 118, 120, 122. The one or more instructions may be instructions that are executable by the controller 110 to perform a specific operation. Some of the commonly known memory implementations may include but are not limited to, flash memory, Random Access Memories (RAMs), and other small package memory types. In some embodiments, the memory 112 can be a data storage device external to the implantable sensor device 100, that includes any of fixed (hard) drives, magnetic tape, floppy diskettes, optical disks, Compact Disc Read-Only Memories (CD-ROMs), and magneto-optical disks, semiconductor memories, such as ROMs, Programmable Read-Only Memories (PROMs), Erasable PROMs (EPROMs), Electrically Erasable PROMs (EEPROMs), flash memory, magnetic or optical cards, cloud computing platforms (e.g. Microsoft Azure and Amazon Web Services, AWS), or other types of media/machine-readable medium suitable for storing electronic instructions. The sensor device 100 can communicate with the aforementioned external data storage device with the network interface 108 and instructions stored within the communication module 120.

The power source 104 can be used to supply electrical power to the implantable sensor device 100 and its associated components and systems. The power source 104 can have a power input connection to receive energy in the form of an electric current and a power output connection to deliver the electric current to the implantable sensor device 100. Further, the power source 104 may be a DC battery or other charging methods. In an embodiment, the power source 104 may use electromagnetic induction to provide electricity to the implantable sensor device 100. For example, the power source 104 can, in some embodiments, have a power input connection that uses wireless charging such that the device 100 can be charged while still implanted within the patient without the need for cables extending through the implantation site. In some embodiments, the power source 104 can include high-capacity body safe batteries which can be charged via electrical input. For example, the batteries can be lithium-ion batteries.

The implantable sensor device 100 can additionally include a base module 114, for performing various operations of the implantable sensor device 100. The base module 114 can facilitate activation of implantable sensor device 100 and monitoring the one or more sensors 106. The base module 114 can include one or more modules such as, but not limited to, a boot-up module 116, a monitoring module 118, a communication module 120, and an action module 122.

The base module 114 can operate directly on the implantable sensor device 100. Alternatively, the base module 114 can operate on an external device 200 connected to the implantable sensor device 100 via the network interface 108 with a wired or wireless connection. The base module 114 can facilitate monitoring the one or more sensors 106 and thus, take an action or provide alerts to the patient or caregivers when the patient is suspected to be incapacitated. The base module 114 may be configured to activate the boot-up module 116, to initialize the implantable sensor device 100. The base module 114 can, for example, be initiated with a power button on the device 100, before the device is implanted into the patient. However, the base module 114 can, in some examples, be initiated remotely after the device has been implanted in the patient.

Upon powering up the device 100, the boot-up module 116 can begin activating the one or more sensors 106 to collect sensor data in vivo. For example, the device 100 can be powered on via the boot-up module 116 for monitoring the health condition of a patient after a surgery performed to place a stent in the patient’s coronary arteries. In use, the boot-up module 116 can activate the sensor array 106 to determine the condition of tissue implanted with the stent. In some embodiments, the boot-up module 116 can be further, or alternatively, configured to calibrate the one or more sensors 106. For example, the boot-up module 116 can calibrate the sensor array 106 by measuring a baseline value and adjusting threshold measurement values. In an embodiment, the one or more sensors 106, or sensor array, can include a temperature sensor which may be calibrated by taking a temperature measurement, receiving a measurement from a thermometer known to be accurate within the same room, comparing the two measurements to determine a correction value, which may simply be the deviation from the known accurate measurement, and then apply the correction value to all future measurements from the now calibrated temperature sensor. The calibration step may be automated or may be performed via manual actions taken by a technician or medical professional. In some devices, multiple measurements may be taken to develop a correction algorithm based upon the multiple measurements. Generally, the calibration step can use a reference source to provide a reliable, known value, though the methods may include other calibration methods.

Further, the boot-up module 116 can be configured to test the network interface 108. For example, the boot-up module 116 can test whether a BLUETOOTH device is working properly by sending a connection request to the Bluetooth device and then by receiving a corresponding acknowledgement for connection confirmation, from the Bluetooth device. Additionally, the boot-up module 116 can configure the implantable sensor device 100. In some embodiments, the boot-up module 116 may set the monitoring parameters to collect the data from the one or more sensors 106. Configuring the implantable sensor device 100 may be completed automatically, or manually via a connected external device 200, such as a mobile device operated by a medical professional, or the patient. For example, the boot-up module 116 can set the level of chemical/ enzyme for collecting the data from the sensor array and establish a threshold value to send a notification to the external device 200.

Further, the boot-up module 116 can confirm that the implantable sensor device 100 is operational. In one embodiment, the boot-up module 116 can confirm the implantable sensor device 100 is in an on-state by monitoring that electrical power consumption of the implantable sensor device 100 is above a certain threshold value, for example 0.8 Watts. In another embodiment, the boot-up module 116 can provide an instruction like a broadcast signal, to the implantable sensor device 100 and looks for an acknowledgement for the broadcast signal, from the implantable sensor device 100.

During the boot up process, the base module 114 can trigger the monitoring module 118. The monitoring module 118, in turn, can be configured to monitor the one or more sensors 106 and store data from the one or more sensors 106. In some embodiments, the data from the one or more sensors 106 may be stored in the memory 112. Alternatively, or additionally, the data collected from the one or more sensors 106 can be stored in a memory of the external device 200 such as a mobile device; the memory 112 can be located locally on the implantable sensor device 100; or the memory 112 may be a cloud server connected to the implantable sensor device 100 through the network interface 108. For example, the monitoring module 118 can monitor the sensor array 106 for chemicals/enzymes and store the value of the chemical/enzyme in the sensor data stored in the memory 112 disposed in the device 100 itself.

The monitoring module 118 can be configured to receive and process data from the sensors 106, in real-time. Additionally, the monitoring module 118 can send the data periodically to the memory 112 at predetermined time intervals. The periodic time intervals may depend upon the type of sensor. For example, the monitoring module 118 can be configured to send the data after every second, to the memory 112. In another example, the monitoring module 118 can be configured to send the data once per day to the memory 112. In some embodiments, the monitoring module 118 can send a value of a measured chemical/enzyme in the tissue around a stent. In an alternative embodiment, the monitoring module 118 can be configured to collect discrete data or waveform data, once every second, from the one or more sensors 106 and store the data in the patient database.

After data are monitored from the one or more sensors 106 by the monitoring module 118, the base module 114 can check whether the data from the monitoring module 118 meet a pre-defined criteria. The pre-defined criteria can be related to validating the data from the monitoring module 118 is a valid or is within a normal, expected, range. For example, in the case where the sensor device 100 is monitoring blood pressure of the patient, the base module 114 can check whether the monitored blood pressure is within normal range, e.g., between 140 mm Hg to 60 mm Hg.

The base module can be programed such that only when the data from the monitoring module 118 satisfies a pre-defined criteria, then the base module 114 triggers the communication module 120. For example, if the patient’s monitored blood pressure is outside of the normal range, the base module 114 can trigger the communications module to contact the external device 200 for alerting a medical professional. The communication module 120 can be configured to send a signal to the external device 200 and receive a response from the external device 200 indicting that the external device 200 is ready to receive the data. For example, the communication module 120 can send a broadcast signal to the external device 200 and receive an acknowledgement from the external device 200, to receive sensor data. The communication module 120 can send the sensed data to the external device 200, in real-time. The communication module 120 may be configured to prioritize and send the data based on the level of importance of the data. For example, the communication module 120 can send data with high priority, e.g., related to heart inflammation in a patient, and the communication module 120 can later send data with low priority e.g., data corresponding to change in blood pressure of a patient. In another example, the communication module 120 can be configured to send data related to a chemical/enzyme measurement that exceeds a threshold value before sending data pertaining to falling blood pressure, having a low priority. In this case, the communication module 120 can send the data related to the chemical/enzyme values that have exceeded the threshold level first then send the data related to falling of the blood pressure below the threshold value.

In some embodiments, the base module 114 can trigger the action module 122 in response to the sensed data. For example, the action module 122 can generate a notification or perform an intervention based upon a received condition or sensor data. In an embodiment, the action module 122 may receive a notification that an implant has been subjected to forces exceeding a predetermined threshold value, which may be a predefined value provided by a manufacturer of the implant. Additionally, or alternatively, the action module 122 may receive data from the one or more sensors 106. The action module 122 can be configured to send a notification to the external device 200 for reporting to the patient or a medical professional. The notification may include an alert, of an abnormal condition, such as a patient’s blood glucose level exceeding a predefined threshold value. The predefined threshold value may be defined by the patient, a medical professional, or determined via an algorithm which may include machine learning. Additionally, a notification may include the measured data.

In some cases, the action module 122 can be configured to perform an intervention such as sending a notification in case the patient is suspected to be incapacitated. For example, the action module 122 may receive data that the patient’s pulse has fallen below a predetermined minimum threshold, such as 40 beats per minute, or the blood-oxygen levels are below a threshold value, such as 70%. Additionally, or alternatively, the action module 122 can be configured to take direct action, e.g., to mitigate risk to the patient 1000 or to save the life of the patient 1000. In some embodiments, the action module 122 can control the implanted device to administer a medication or provide electrostimulation to the patient. For example, the sensors 106 may sense a patient’s blood sugar is above a predetermined value, such as 150, and may direct an insulin delivery device to administer a prescribed amount of insulin. The dose may be a fixed amount determined by a user or medical professional, or may be determined via an algorithm based upon the measured data.

In another use case, the action module 122 can be configured to deactivate the implanted sensor device 100 if a failure of the implanted device is detected. For example, the action module 122 can deactivate a stent placed in the artery of a patient if a leakage or failure is detected in the stent implanted inside the artery. Additionally, or alternatively, the action module 122 may be configured to activate a redundant device. For example, a stent may be comprised of two channels where only one is in use. Upon receiving sensor data indicating a blockage, either due to a drop or increase in pressure or an absence of blood flow, the second channel may be opened to allow blood to flow. In some embodiments, the stent can include a strain or fluid flow resistance sensor to ensure that the stent is open and a sufficient flow rate is passing through the sten. In an alternate embodiment, two pacemakers may be installed. One can be preconfigured to be in an active state, while the second device is in a passive state. The second device may include a means of monitoring the output of the first device, activating when insufficient output is detected or if the first device fails to activate when required.

In further embodiments, two blood glucose sensors may be installed, each monitoring a patient’s blood sugar. The two sensors may be in communication with each other and an insulin delivery device. When a first sensor determines insulin is required, the first sensor may instruct the insulin delivery device to administer insulin to the patient. The first sensor may additionally communicate to the second sensor that insulin has been requested or delivered and instruct the second sensor to defer any requests for additional insulin for a predefined period of time, e.g., for at least 30 minutes. In this example, either the first sensor or the second sensor may act as the primary device, instructing the delivery of insulin, depending on the measurements acquired by the sensor. In addition, or alternatively, the insulin delivery device can include a delivery valve and a sensor for monitoring proper drug delivery. For example, the sensor can sense that a proper fluid flow rate is measured exiting the delivery valve when insulin is required and halting delivery of the insulin if the flow rate is too high. Additionally, or alternatively, the communication module 120 can notify a medical professional that there may be an error with the drug delivery valve and medical intervention may be required.

In some examples, the action module 122 can be programed to perform certain therapeutic functions to the patient upon reaching certain sensed condition from the sensor 106. The one or more sensors 106 may correspond to sensors capable of measuring and monitoring heart rate, heart rhythm, stroke volume, and perfusion. For example, a neurostimulator may be surgically implanted to deliver mild electrical impulses to nervous tissues through one or more thin wires, called leads inserted into tissue of the patient upon detection of sensed data outside of a predetermined threshold. In another embodiment, a pacemaker may be used to maintain heart rhythm.

As noted above, the device 100 can include a network interface to allow the device 100 to communicate with an external device 200. The network interface 108 can be used to exchange data between the implantable sensor device 100 and one or more external devices 200, in real time. The network interface 108 may include a BLUETOOTH, ZIGBEE, Wi-Fi, 5G, 4G LTE, 3G, NFC, or other wireless communication modules. Additionally, or alternatively, the network interface 108 can include a wired communication interface for a wired connection with external devices 200. The network interface 108 can transmit data sensed by the sensors 106 to an external device 200 such as a desktop computer, laptop, mobile computing device, tablet computer, smartphone, smart speaker, or other I/O computing devices. In one example, the external device 200 may be connected to the device 100 via near-field communication (NFC). NFC can utilize less power than other transmission methods and its limited range decreases the likelihood of interception of potentially sensitive data. In some embodiments, a cellular service such as 5G may be required to facilitate communication with a remote device, such as a mobile device belonging to the patient’s physician or caregiver. Additional embodiments may include multiple network interfaces 108, such as utilizing NFC to communicate with local devices, such as other sensors, implants, or a patient’s mobile device, and 5G for communication with remote devices, such as those belonging to a medical professional or caregiver. The network interface 108 can be connected continuously to the external device 200 via the network interface 108. Alternatively, the network interface 108 can be connected intermittently to the external device 200 to save power in use cases where continuous data transfer is not necessary. In case of intermittent connection, the network interface 108 may store data in its internal buffer, or on the memory 112, and send data to the external device 200 at a regular, predefined, intervals of time. In some embodiments, the network interface 108 can be connected on demand or when polled by the external device 200 (via Radio-frequency identification - RFID or Near-Field Communication - NFC) and only be activated upon receiving power from the external device 200 via inductance, or other signal types.

In some examples, the network interface 108 can additionally, or alternatively, connect to a cloud application such as distributed computing network. Distributed computing can assist in performing operations related to the data received from the one or more sensors 106, by passing the data from the one or more sensors 106, over the cloud, via the network interface 108. The distributed computing network can additionally provide for redundant backup of the data to prevent loss of critical health information from the patient.

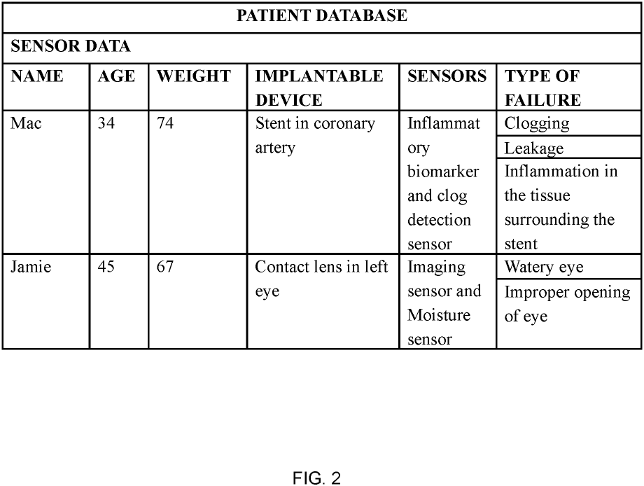

As discussed herein, the sensor device 100 collects data from the patient, the collected data can be stored as a patient database. FIG. 2 illustrates the patient database which stores data for the implantable sensor device 100, according to an embodiment. The patient database can be stored locally, on the device 100, or can be transmitted with the network interface 108 to the external device 200. The patient database may be configured to store the sensor data, data related to implants, and software modules. In an embodiment, the sensor data may include at least but not limited to name, age, weight, implantable device, sensors, and type of failure for a particular patient. Further, the patient database may store instruction sets or programming of the modules like a boot-up module 116, a monitoring module 118, a communication module 120, and an action module 122. The patient database can be formatted to be easily integrated into virtual, or electronic, medical charts for patients for easy retrieval of the data by medical professionals.

In the illustrated embodiment of FIG. 2, the sensor data includes data for a first patient named Mac, who is 34 years old and weighs 74 kilograms, and who was operated on to implant stent in his coronary artery. Further, the implantable sensor device 100 used in Mac includes a clogging detection sensor and inflammatory biomarkers to monitor the stents and respective failures in order to determine the condition of Mac post-surgery. Surgical failures can include clogging, leakage, and inflammation due to the stent implanted in the coronary artery.

The table of FIG. 2 can include data for multiple patients with multiple and varied sensor types. For example, the sensor data may include data for a second patient named Jamie, who is 45 years old and weighs 67 kilograms. The patient database can indicate that Jamie was operated on to implant a contact lens in his left eye. In this case, the implantable sensor device 100 can use the sensor assembly including an imaging sensor to monitor the opening of the eye and a moisture sensor for monitoring the moisture in the eye of Jamie, post-surgery. The patient database can outline failures that include at least watery eye or improper opening of the eye.

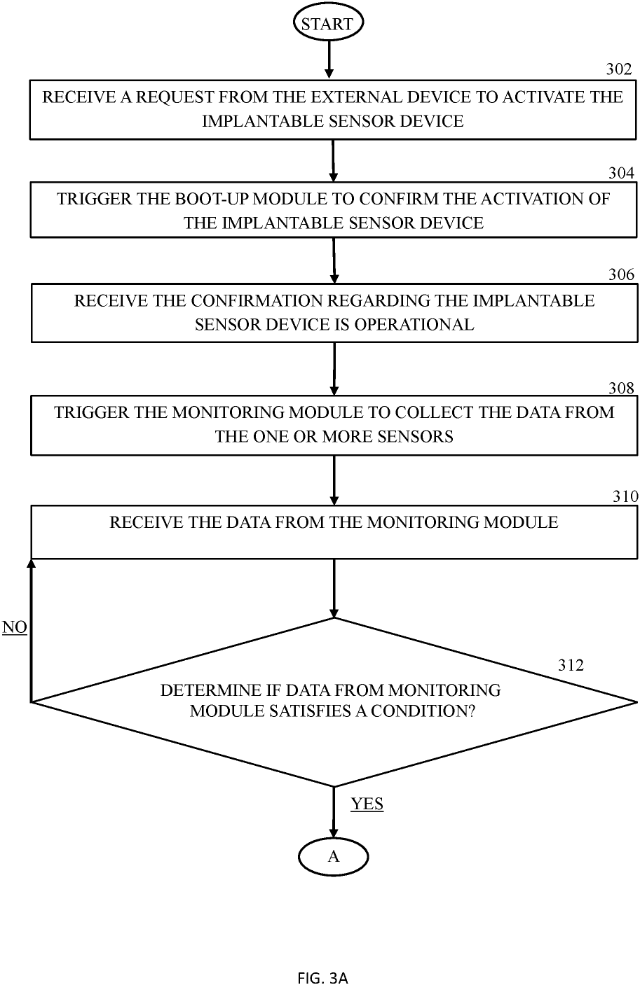

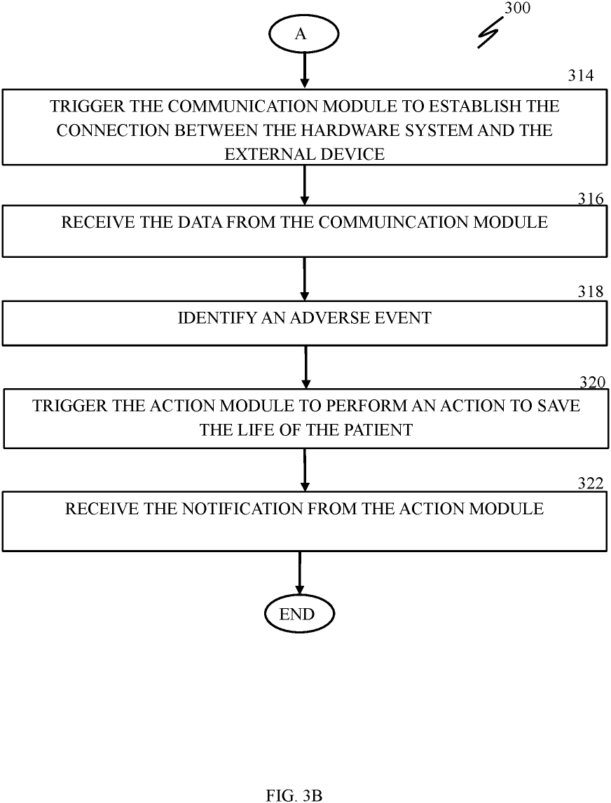

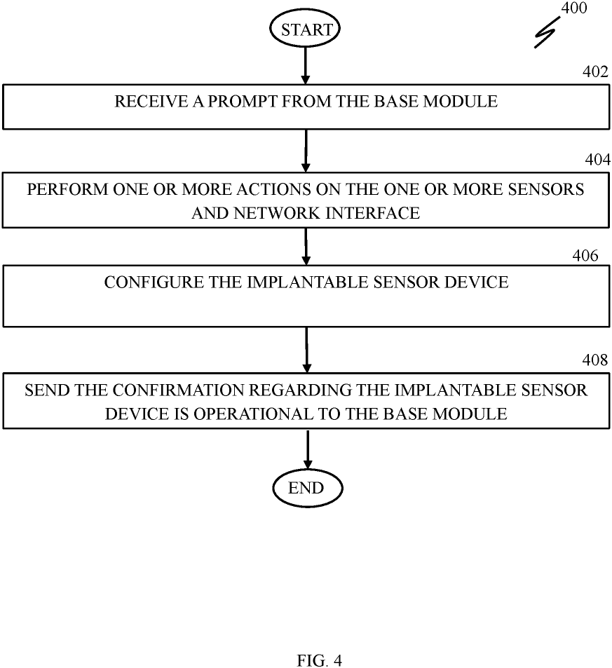

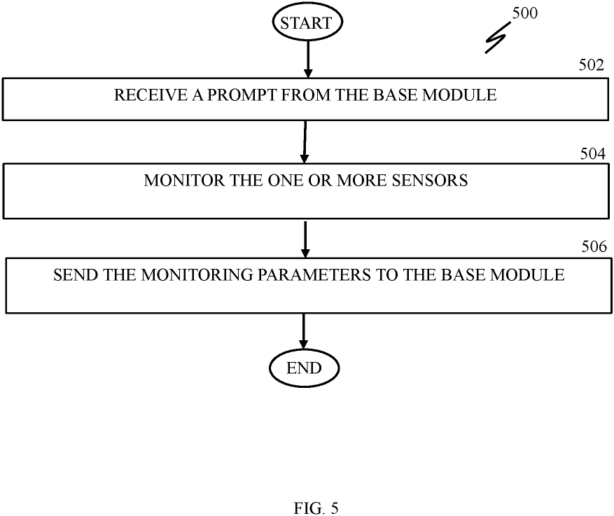

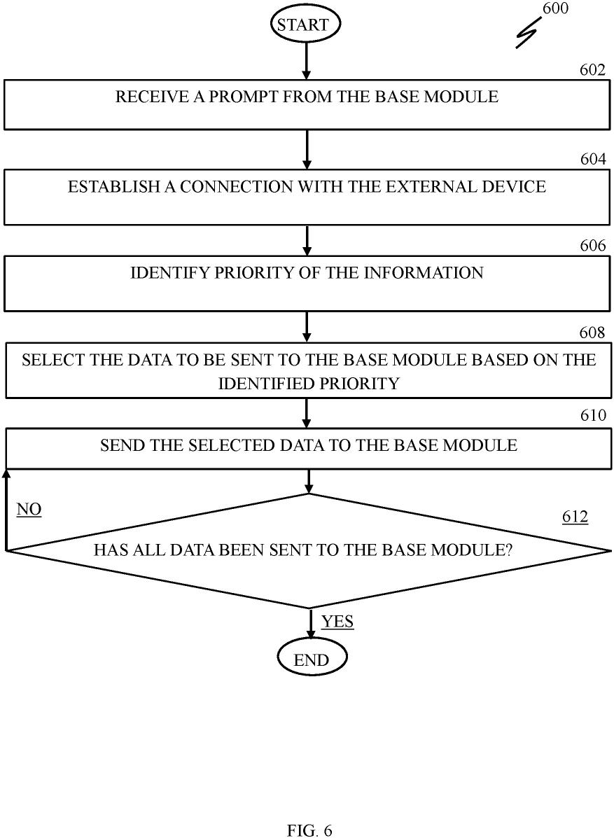

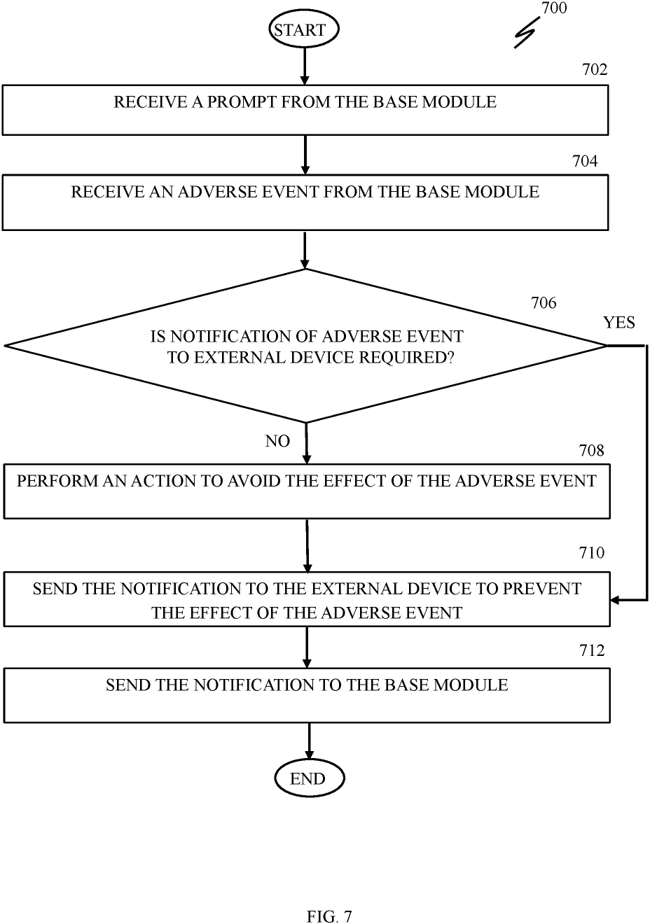

FIG. 3A and FIG. 3B illustrate an operation of the base module 114, shown as a flowchart. The embodiment of FIG. 3A and FIG. 3B are explained herein in conjunction with FIG. 4, FIG. 5, FIG. 6, and FIG. 7. In certain implementations, the functions noted in the illustrated blocks may occur in an order other than those shown in the figures. For example, two blocks are shown in succession in FIG. 3A and FIG. 3B may be executed substantially concurrently or the blocks may sometimes be executed in the reverse order, depending upon the functionality involved. In addition, the process descriptions or blocks in flow charts should be understood as representing decisions made by a hardware structure such as a state machine, or the controller 110 of the implantable sensor platform 102.

The base module 114 may receive a request from the external device 200 to activate the implantable sensor device 100, at step 302. For example, the base module 114 can receive a request sent from a device of the surgeon of the patient to activate the in vivo sensor array to monitor a stent placed in the patient’s coronary artery. The base module 114 can trigger the boot-up module 116 to confirm the activation of the implantable sensor device 100, at step 304. The base module 114 can send a signal to the external device 200 requesting activation to confirm the activation of the implantable sensor device 100, at step 304. Further, to confirm the activation, the boot-up module 116 may activate and calibrate the one or more sensors 106, test the network interface 108, and configure the implantable sensor device 100. Upon activation and calibration of the one or more sensors 106, test the network interface 108, and configure the implantable sensor device 100, the external activation device can receive confirmation regarding the implantable sensor device is operational, at step 306. The boot-up module 116 may configure the implantable sensor device 100 with regards to the monitoring parameters such as to when to store the health data and when to send the notification to the external device 200. The boot-up module 116 can trigger the monitoring module 118 to begin to collect data from the one or more sensors 106, at step 308, and can then receive such data, at step 310. For example, the boot-up module 116 can configure the implantable sensor device 100 to report the blood pressure i.e., 120 mm Hg to 20 mm Hg of the patient at regular intervals. In an embodiment, the health data may be stored in the patient database (as shown in FIG. 2), and a notification may be sent to an external device depending on a measured value of one of the monitoring parameters. In one case, if one of the monitoring parameters reaches a threshold value, the implantable sensor device 100 can send a notification to the external device 200 for reporting to the medical professional, such as the surgeon. Advantageously, the monitoring module 118 can determine if the monitoring parameters have reached a threshold value, at step 312. If the monitoring module 118 determines that the monitoring parameters are within the acceptable range, then the monitoring module 118 can continue to receive and transmit the data to the external device 200 at the predetermined intervals, at step 310. If the monitoring module 118 determines that the monitoring parameters are outside the acceptable range, at step 312, the communication module 120 can be triggered to establish a connection between the device 100 and the external device 200.

In another example, the boot-up module 116 can configure the implantable sensor device 100 to store the data related to an implanted stent and the device 100 can send a notification to the patient’s surgeon in case of a detected clot. The stent may be monitored by one or more pressure sensors which may be integrated into the stent. An increase in pressure at the inlet of the stent or a drop in pressure at the outlet of the stent may indicate the presence of a clot or other flow restriction. Alternatively, a flow sensor may be integrated into a stent to monitor the flow of blood directly. In another embodiment, the health data may be stored and notification may be sent to the external device 200 after a predetermined period of time. For example, the boot-up module 116 can activate and calibrate the sensor array 106 and configure the implantable sensor device 100 to store the amount of inflammation of the tissue around the area of stent placement as indicated by increased pressure detected by the sensor and send a notification to the surgeon once a day about the level of inflammation.

The operation of the boot-up module 116 is explained in further detail with respect to FIG. 4. FIG. 4 illustrates the operation of the boot-up module 116, as shown in flowchart 400, according to an embodiment. It should also be noted that in some alternative implementations, the functions noted in the blocks may occur out of the order noted in the drawings. For example, two blocks shown in succession in FIG. 4 may be executed substantially concurrently or the blocks may sometimes be executed in the reverse order, depending upon the functionality involved. In addition, the process descriptions or blocks in flow charts should be understood as representing decisions made by a hardware structure such as a state machine.

As discussed with respect to FIG. 3A, the boot-up module 116 can receive a prompt from the base module 114 to confirm the implantable sensor device 100 is active, at step 304. Looking to FIG. 4, at step 402, the boot-up module 116 receives a prompt from the base module 114 and can perform one or more successive actions on the one or more sensors 106 and network interface 108, at step 404. The one or more actions on the one or more sensors 106 may include activating and calibrating the one or more sensors 106. Sensor calibration can be an adjustment, or a set of adjustments, performed on a sensor to improve the sensors accuracy. For example, an implanted pacemaker may be automatically calibrated by an electronic lead detecting electrical impulses emitted by the pacemaker, comparing the measured value to an expected value, and determining a correction value. The correction value may then be applied to future measured values to improve the accuracy of those measurements. The correction value may be a simple difference between a measured and expected value, or may be a model based on multiple measurements, such as by emitting multiple impulses of varying power and comparing each measured value to an expected value for each power level. In some embodiments, the calibration may occur in situ. In other embodiments, the calibration occurs prior to implantation. For example, a blood glucose sensor may be used to measure a solution of known glucose concentration, and a correction factor applied to compensate for a variation in the measurement from the known concentration. Further, it should be noted that the error in sensor measurement can occur due to improper zero reference, shift in sensor’s range, mechanical wear, or damage. The boot-up module 116 may perform the calibration to avoid these errors. The one or more actions on the network interface 108 may include performing a test for troubleshooting the bandwidth and latency issues associated with the network interface 108.

In addition to calibrating the one or more sensors 106, the boot-up module 116 can perform actions on the network interface 108, at step 404. For example, information related to bandwidth and latency may be used to prioritize data, via the network interface 108. Successively, the boot-up module 116 can configure the implantable sensor device 100, at step 406. The boot-up module 116 may configure the implantable sensor device 100 monitoring parameters including when to store the health data and send data to an external device 200. For example, the boot-up module 116 can configure the implantable sensor device 100 to store the data related to the amount of leakage of blood due to stent and send a notification to the surgeon in case of a sensed clot. In another embodiment, the health data may be stored and a notification may be sent to the external device 200 after a certain period of time. For example, the boot-up module 116 may activate and calibrate the sensor array 106 and configure the implantable sensor device 100 to store the amount of inflammation of the tissue around the area of stent placement and send a notification to the surgeon once in a day about the inflammation. Thereafter, the boot-up module 116 may send the confirmation regarding the implantable sensor device 100 is operational to the base module 114, at step 408.

Returning to FIG. 3A and FIG. 3B, the base module 114 can receive the confirmation that the implantable sensor device 100 is operational, from the boot-up module 116, at step 306. In some embodiments, the base module 114 can additionally, or alternatively, receive the one or more monitoring parameters from the boot-up module 116. The base module 114 may trigger the monitoring module 118 to collect the data from the one or more sensors 106 based on the received monitoring parameters, at step 308. The monitoring module 118 can monitor the one or more sensors 106 continuously and store the data received from the one or more sensors 106 based on the configured parameter of the implantable sensor device 100. In an embodiment, the monitoring module 118 may receive data periodically, at step 310. For example, the monitoring module 118 can receive data once per second from an inflammatory biomarker, blood pressure sensor, and clog detection sensor. In another embodiment, the monitoring module 118 can receive data once per day from the inflammatory biomarker, blood pressure sensor, and clog detection sensor.

FIG. 5 illustrates the operation of the monitoring module 118, shown as flowchart 500, according to an embodiment. In some alternative implementations, the functions noted in the blocks may occur out of the order noted in FIG. 5. For example, two blocks shown in succession in FIG. 5 may be executed substantially concurrently or the blocks may sometimes be executed in the reverse order, depending upon the functionality involved. In addition, the process descriptions or blocks in flow charts should be understood as representing decisions made by a hardware structure such as a state machine.

As shown in FIG. 5, the monitoring module 118 receives a prompt from the base module 114 to initialize the monitoring module 118 and prepare to monitor the one or more sensors 106, at step 502. The prompt may include instructions that the sensors are to be monitored and may further comprise threshold values for a sensor indicating when to send the data to the base module. In some embodiments, the data may be communicated to the base module in real time. In alternate embodiments, the monitoring module 118 may monitor a sensor 106 until a threshold value has been received. The prompt may additionally comprise a correction factor to be applied to data measured by the sensors. It can be noted that the monitoring module 118 may be triggered to monitor the one or more sensors 106 and enable the storage of the data from the one or more sensors 106. Successively, the monitoring module 118 may monitor the data collected from the one or more sensors 106, at step 504. For example, the one or more sensors 106 can monitor that a patient has a blood pressure of systolic 120 mm Hg and diastolic 80 mm Hg. The monitoring module 118 may monitor the one or more sensors 106 continuously and store the data received from the one or more sensors 106 based on the configured parameter of the implantable sensor device 100. In some embodiments, the monitoring module 118 may receive data periodically. In an embodiment, the monitoring module 118 receives data once per second from an inflammatory biomarker, a blood pressure sensor, or a clog detection sensor. Alternatively, or additionally, the monitoring module 118 can receive data once per day from an inflammatory biomarker, a blood pressure sensor, or a clog detection sensor. In an embodiment, the monitoring module 118 may receive data from the inflammatory biomarker, blood pressure sensor, and clog detection sensor, at pre-defined time intervals set by the doctor. The monitoring module 118 can send the data received from the one or more sensors 106 to the base module 114, at step 506. For example, the monitoring module 118 can send data showing that the patient has a blood pressure of systolic 120 mm Hg and diastolic 80 mm Hg, to the base module 114.

As shown in FIG. 3A, the base module 114 can receive the data from the monitoring module 118, at step 310. The base module 114 may determine whether the received data from the monitoring module 118 satisfies a pre-defined condition, at step 312. For example, the base module 114 can determine that the blood pressure received from the monitoring module 118 is valid value, or lies within a normal limit, for blood pressure for a human being i.e., 140 mm Hg to 60 mm Hg. In one case, if the data received from the monitoring module 118 does not satisfy the pre-defined condition, then the base module 114 may return to step 310 to receive data from the monitoring module 118. In another case, if the data received from the monitoring module 118 satisfies the pre-defined condition, the base module 114 moves to step 314 of FIG. 3B. For example, the base module 114 can determine that the blood pressure received for a patient, i.e., 120 mm Hg and diastolic 80 mm Hg, is a valid blood pressure reading and lies within normal blood pressure range of 140 mm Hg to 60 mm Hg. The pre-defined conditions may be variable depending on the time since the procedure took place or changed if a doctor determines that closer monitoring is required. Such a modification of the pre-defined conditions can be performed by sending an update from the external device 200 to the implanted sensor device 100 through the network interface 108.

Depending on the sensed data from the one or more sensors 106, the base module 114 may trigger the communication module 120 to establish a connection between the implantable sensor device 100 and the external device 200, at step 314. In some embodiments, the communication module 120 may establish a connection with the external device 200 through open channels. In an embodiment, the communication module 120 may send the information related to the blood pressure to the external device 200 using ubiquitous radio broadcast. In another embodiment, the communication module 120 may establish a connection with the external device 200 on a dedicated channel. In an embodiment, the communication module 120 may send the information related to the blood pressure to the external device 200 using a Bluetooth device. It can be noted that the operation of the communication module 120 is explained in regard to FIG. 6.

FIG. 6 illustrates the general operation of the communication module 120, as shown in flowchart 600, according to an embodiment. In some alternative implementations, the functions noted in the blocks may occur out of the order noted in the drawings. For example, two blocks shown in succession in FIG. 6 may be executed substantially concurrently or the blocks may sometimes be executed in the reverse order, depending upon the functionality involved. In addition, the process descriptions or blocks in flow charts should be understood as representing decisions made by a hardware structure such as a state machine.

The flowchart 600 shows the communication module 120 receiving a prompt from the base module 114, at step 602. The communication module 120 can, for example, upon receiving the prompt from the base module 114, establish a connection between the network interface 108 and the external device 200, at step 604. The connection can be wired or wireless. In an embodiment, the communication module 120 can establish a connection with the external device 200 through open channels. Alternatively, or additionally, the communication module 120 may establish the connection with the external device 200 by sending a connection request to the external device 200 and then receiving a corresponding acknowledgement for connection confirmation from the external device 200. The communication module 120 may identify priority of information, at step 606. For example, the priority of information may be identified based on a pre-defined priority order, such as defining any sudden infection or allergic reaction during the surgical procedure, inflammation around the surgical site, and change in blood pressure of the patient as being high priority, while sending data of nominal sensed conditions as low priority. For example, the communication module 120 can identify the priority of information related to the patient as follows, an inflammatory reaction next to the heart, at priority 1, and the blood pressure of 120 mm Hg and diastolic 80 mm Hg, at priority 2.

Based on the identified priority, the communication module 120 may select the data to be sent to the base module 114, at step 608. For example, based on the identified priority, the communication module 120 selects the information related to the inflammation next to the heart of the patient at priority 1, to be sent to the base module 114 first. The communication module 120 can send the selected data to the base module 114, at step 610. For example, the communication module 120 can send the selected information related to the priority 1 information, being the inflammation next to heart of the patient, to the base module 114, then send the lower priority information at a later time.

Once the data has been sent by the communications module, the communication module 120 may determine whether all the data has been sent to the base module 114, at step 612. Step 612 provides for a check to ensure that the fidelity of the data being sent is as complete as possible so that the doctor is not making medical decisions based on an incomplete picture. In one case, when the communication module 120 determines that all the data has not been sent to the base module 114, then the communication module 120 may move to step 606 to identify the priority of the remaining data and repeat the steps 606 to step 612, till all the data is sent to the base module 114. For example, after sending the information related to inflammation next to the heart of the patient, the communication module 120 can determine that the information related to the blood pressure readings of the patient has not been sent. Thus, the communication module repeats steps 606 to 612 to send the data to the base module 114. In another case, when the communication module 120 determines that all the data is sent to the base module 114, then the process, of FIG. 6, can end.

Referring back to FIG. 3B, the base module 114 can receive data from the communication module 120, at step 316. Based on the received data, the base module 114 may identify an adverse event if the data exceeds predetermined thresholds, at step 318. In some embodiments, the adverse event may be identified by comparing the sensor data to the threshold value. For example, the sensors 106 can sense that the patient’s heart has an inflammation of degree 4 as compared to a pre-defined threshold value of inflammation of degree 2 and the base module can therefore identify the adverse effect of inflammation.

In another embodiment, the adverse event may be identified by, identifying trends in data stored in the patient data. For example, a constant inflammation of 4 seconds may enable the base module 114 to identify the adverse effect on the patient’s heart or surrounding tissue. Alternatively, or additionally, the adverse event may be identified by using machine learning (ML) or artificial intelligence to identify an adverse event, based on previous or historical sensor data related to the patient. For example, the base module 114 can identify, based on inflammation next to the patient’s heart, a lethal stroke due to a clot near the implanted stent can be expected, with the help of ML algorithm. In some embodiments, the ML algorithms can include algorithms that use sensor data to predict and/or diagnose adverse events include. For example, those disclosed in Non-invasive diagnosis of deep vein thrombosis from ultrasound imaging with machine learning - Kainz et al https://www.nature.com/articles/s41746-021-00503-7; Somani, S.S. et al., Development of a machine learning model using electrocardiogram signals to improve acute pulmonary embolism screening, European Heart Journal, Nov. 25, 2021, DOI: 10.1093/ehjdh/ztab101; Using machine learning to identify clotted specimens in coagulation testing, Fang et al https://pubmed.ncbi.nlm.nih.gov/33660491/; and/or A prehospital diagnostic algorithm for strokes using machine learning: a prospective observational study https://www.nature.com/articles/s41598-021-99828-2.

In some embodiments, the adverse event may be related to the failure of the implantable sensor device 100. In another embodiment, the adverse event may be created by the one or more sensors 106 which is made up of biodegradable material by creating unexpected data. For example, as the biodegradable sensors 106 are broken down due to age of use, the sensors 106 may no longer provide valid, reliable, data. In such a case, the doctor or surgeon may need to determine if the sensors 106 need to be replaced or if the patient has sufficiently recovered to suspend data sensing. Additionally, or alternatively, the adverse event may be created by the one or more sensors 106 in the absence of the calibration of the one or more sensors 106. In such a case, the implantable sensor device 100 can determine that a recalibration of the one or more sensors 106 is required.

Upon sensing an adverse event, the base module 114 can trigger the action module 122 to perform an action, at step 320. In an embodiment, the action module 122 may control certain modules in the implanted device. For example, the action module 122 may send a control signal to dilate a stent while retaining the clot to permit blood flow to avoid lethal strokes, heart attacks, or pulmonary embolisms. In another embodiment, the action module 122 can send a notification to the external device 200 about the monitoring parameters to notify a medical professional. For example, the communication module 120 may send a notification to the surgeon about the escaped clot in the coronary artery. It can be noted that the operation of the action module 122 is explained in FIG. 7.

FIG. 7 illustrates an algorithm for the operation of the action module 122, as shown in flowchart 700, according to an embodiment. It should also be noted that in some alternative implementations, the functions noted in the blocks may occur out of the order noted in the drawings. For example, two blocks are shown in succession in FIG. 7 may be executed substantially concurrently or the blocks may sometimes be executed in the reverse order, depending upon the functionality involved. In addition, the process descriptions or blocks in flow charts should be understood as representing decisions made by a hardware structure such as a state machine.

In some embodiments, the action module 122 can additionally receive a prompt from the base module 114, at step 702. The action module 122 can receive a notification of a determined adverse event from the base module 114, at step 704. For example, the action module 122 can receive notification of the adverse event from the base module 114 that the inflammation of degree 4 next to the patient’s heart is adverse as compared to a pre-defined threshold value of inflammation of degree 2. The action module 122 can receive the notification in real time as the base module is monitoring the inflammatory biomarker, blood pressure sensor, and clog detection sensor sensing the parameters of the stent placed in patient’s coronary artery. Additionally, or alternatively, the adverse event may be caused by a software or hardware error of the implanted device. For example, the implanted stent may not be performing as expected such that there is not sufficient blood flow through the stent passage, leading to blockage in artery.

The action module 122 may check if notification of the adverse event to the external device 200 is required, at step 706. In one case, when the notification to the external device 200 is not required as the action module 122 may take an appropriate action to avoid the effect of the adverse event, at step 708. For example, when the notification to the external device 200 is not required, the action module 122 can shut off the implanted device to prevent more adverse events from occurring. In another embodiment, the action module 122 may enable the implanted device to perform a system diagnostic and reactivate to prevent adverse events. For example, a first pacemaker may be deactivated due to insufficient output and a redundant, second pacemaker may be activated to take over for the first pacemaker. The first pacemaker may then perform a calibration operation, wherein the second pacemaker may become inactive for a few seconds while the primary pacemaker emits electrical impulses which are measured. The measured output of the first pacemaker can be analyzed and a correction factor determined, while the second pacemaker resumes operation. The second pacemaker can then be deactivated, allowing the first pacemaker to resume operation provided the now corrected output is within operational ranges.

In some scenarios, the action module 122 can determine that a notification needs to be sent to the external device 200 to prevent adverse events and the action module 122 may send the notification to the base module 114, at step 710. In an embodiment, the action module 122 can send a notification to an external device 200, e.g., a mobile device, upon receiving a sensed blood pressure level of the patient has reached 80 mm Hg, which is far below the predefined threshold i.e., 110 mm Hg. The action module 122 can contact the surgeon or emergency services, in that case. The action module 122 may send any of a text message, email, voice message, audible notification, or other electronic communication. Thereafter, the action module may send a notification to the base module 114, at step 712. Consequently, the base module 114 may receive a receipt of the action performed by the action module 122, at step 322. In some embodiments, if a receipt is not received, another attempt may be made to send a notification.

It will be appreciated by those skilled in the art that changes could be made to the embodiments described above without departing from the broad inventive concept thereof. It is to be understood, therefore, that this disclosure is not limited to the particular embodiments disclosed, but it is intended to cover modifications within the spirit and scope of the subject disclosure as disclosed above.

What is claimed is:

1. An implantable sensor device comprising:
at least one sensor configured to monitor temperature or pH at a site;
a memory;
a controller;
a base module,
an action module; and
a network interface;
wherein the implantable sensor device is configured to:
sense and monitor the temperature and the pH at the site, with the at least one sensor to collect monitored data;
store the monitored data in the memory;
monitor the stored data for an adverse event including an infection as a function of the temperature and the pH; and
perform an action in response to the adverse event including sending a control signal to the action module to deliver a medication at the site by the implantable sensor.

2. The implantable sensor device of claim 1, wherein the base module is further configured to confirm activation of the implantable sensor device.

3. The implantable sensor device of claim 1, wherein the base module is further configured to establish a data connection with an external device, via the network interface.

4. The implantable sensor device of claim 3, wherein the network interface is configured to send the monitored data to the external device, wherein the monitored data is sent based on the configuration of the implantable sensor device.

5. The implantable sensor device of claim 3, wherein the data connection is a wireless network connection between the implantable sensor device and the external device.

6. The implantable sensor device of claim 1, wherein the base module is configured to send a control signal to the action module to mitigate an impact of the adverse event including alerting a medical professional.

7. The implantable sensor device of claim 6, wherein the action module is configured to perform a medical procedure to mitigate the impact of the adverse event.

8. The implantable sensor device of claim 1, wherein the base module is configured to send the monitored data, in real-time, via the network interface to an external device.

9. The implantable sensor device of claim 1, wherein the at least one sensor is at least one of a force transducer, a strain gauge, temperature sensor, pH sensor, or an accelerometer.

10. The implantable sensor device of claim 9, wherein the at least one sensor includes two or more sensors of different types.

11. A system for monitoring implant conditions in vivo, the system comprising:
an implantable medical device;
an external monitoring computing device; and
an implantable sensor, disposed on the implantable medical device, including at least one sensor configured to monitor temperature or pH at a site, a memory, a controller, and a base module,
wherein the base module is configured to:
monitor the implantable sensor with the at least one sensor to collect monitored data, the monitored data including temperature and pH at the site;
store the monitored data in the memory;
monitor the stored data for an adverse event including infection as a function of the temperature and the pH; and
perform an action in response to the adverse event including delivering a medication with the implantable sensor to the site.

12. The system of claim 11, wherein
the base module further includes a network interface, and
the base module is further configured to establish a data connection with the external monitoring computing device, via the network interface, to report the monitored conditions of the implanted medical device.

13. The system of claim 12, wherein the data connection is a wireless network connection between the implantable sensor and the external monitoring computing device.

14. The system of claim 11, wherein the base module is configured to send a control signal to mitigate an impact of the adverse event including alerting a medical professional.

15. The system of claim 14, wherein the base module is configured to perform a medical procedure on a patient to mitigate the impact of the adverse event.

16. The system of claim 11, wherein the base module is configured to send the monitored data, in real-time, to the external monitoring computing device.

17. The system of claim 11, wherein the at least one sensor is at least one of a force transducer, a strain gauge, temperature sensor, pH sensor, or an accelerometer.

18. The system of claim 17, wherein the at least one sensor includes two or more sensors of different types.

19. The system of claim 11, wherein the at least one sensor is biodegradable.

20. A method of in vivo monitoring, the method comprising,
implanting an implantable sensor at a site, the implantable sensor including at least one sensor, a memory, a controller, and a base module,
sensing at least one physical attribute at the site including one of temperature or pH with the at least one sensor to collect monitored data;
storing the monitored data in the memory;
monitoring the stored data for an adverse event including infection as a function of the temperature or the pH; and
performing a corrective action in response to the adverse event including delivering, with the implantable sensor, a medication at the site.

21. The method of claim 20, wherein the implanting step includes implanting an implanted device and the implantable sensor is disposed on the implanted device.

22. (canceled)

23. The method of claim 20,
wherein the sensing step includes sensing at least two physical attributes.

24. The method of claim 23, further comprising identify priority of the at least two physical attributes based upon a pre-defined priority order and prioritizing a higher priority sensed physical attribute over a lower priority sensed physical attribute.

25. The method of claim 24, wherein the higher priority sensed physical attribute is sent to an external computing device.

26. The method of claim 20, further including establishing a wireless data connection with an external monitoring computing device, via a network interface, to report the monitored data.

27. (canceled)

28. The method of claim 20, further including send the monitored data, in real-time, to an external monitored computing device.

29. The method of claim 20, wherein the at least one sensor is at least one of a force transducer, a strain gauge, temperature sensor, pH sensor, or an accelerometer.

30. The method of claim 20, wherein the at least one sensor includes two or more sensors of different types.
